(19) United States
(12) Patent Application Publication
(10) Pub. No.: US 2024/0336651 A1
(43) Pub. Date: Oct. 10, 2024

(54) Method for Manufacturing Biosensor

(71) Applicant: NATIONAL SUN YAT-SEN UNIVERSITY, Kaohsiung City (TW)

(72) Inventors: Hong-Wei Yang, Kaohsiung City (TW); Nan-Si Li, Kaohsiung City (TW); Ying-Pei Hsu, Kaohsiung City (TW); Hao-Han Pang, Kaohsiung City (TW)

(21) Appl. No.: 18/748,165

(22) Filed: Jun. 20, 2024

Related U.S. Application Data
(62) Division of application No. 17/483,840, filed on Sep. 24, 2021.
(60) Provisional application No. 63/083,872, filed on Sep. 26, 2020.

(51) Int. Cl. C07K 2/00; A61K 38/16; B82Y 40/00; C03C 17/32; C07K 14/005; G01N 21/78; G01N 27/414; G01N 33/543

(52) U.S. Cl. CPC C07K 2/00; A61K 38/162; C03C 17/32; C07K 14/005; G01N 21/78; G01N 27/4145; G01N 33/54393; B82Y 40/00; C03C 2217/70; C07K 2319/30; C07K 2319/735; C07K 2319/74; C12N 2795/00023

(57) ABSTRACT

A method for manufacturing a glass-based biosensor is used to solve the problem of the use of a solution containing a strong acid or a strong base or of an oxygen plasma treatment. The method comprises modifying a silicon-containing substrate by an alcohol solution to form negative charges on at least one coupling surface of the silicon-containing substrate. A least one active layer of polymer having positive charges is formed on the at least one surface of the silicon-containing substrate, respectively. Each of the at least one active layer of polymer has a coupling surface and an active surface opposite to the coupling surface, and the at least one active layer of polymer couples to the silicon-containing substrate via the coupling surface. A plurality of capture biomolecules couples to the active surface.

### CROSS REFERENCE TO RELATED APPLICATIONS

This is a divisional application of U.S. patent application Ser. No. 17/483,840 filed on Sep. 24, 2021. The application claims the benefit of U.S. provisional application No. 63/083,872, filed Sep. 26, 2020, and the entire contents of which are incorporated herein by reference.

### BACKGROUND OF THE INVENTION

### 1. Field of the Invention

The present invention generally relates to a method for manufacturing a biosensor and, more particularly, to a method for manufacturing a biosensor in which fewer waste is produced.

### 2. Description of the Related Art

In order to evaluate whether a suspected patient is infected by a virus, generally, the quantitative real-time polymerase chain reaction (RT-qPCR) method can be used to detect the virus with a specific primer pair. However, not only the quantitative real-time polymerase chain reaction method with high specificity and high sensitivity requires complicated specimen pre-processing procedures, expensive laboratory equipment, and the workers to carry out the quantitative real-time polymerase chain reaction method also need complete training. Therefore, it is inconvenience for point-of-care (POC) of specific virus by the quantitative real-time polymerase chain reaction method.

In order to perform real-time detection, a conventional optical biosensor that can be used with a smartphone to detect molecules or cancer biomarkers in body fluids has been developed. If a specimen contains an antigen of specific virus, and the antigen that can specifically bind to the antibody provided on the surface of the conventional optical biosensor, the optical signal generated by the specific binding of the antibody to the antigen can be detected by the smart phone, confirming that the specimen has infected by the specific virus.

However, in the method for manufacturing the conventional optical biosensor, a silicon-containing substrate surface with negative charges is formed by a strong acid solution or a strong alkali solution (for example, an aqueous sodium hydroxide solution). An active polymer layer with positive charges is then electrostatically bond to the surface of the silicon-containing substrate, and an antibody with negative charges can electrostatically bond to the surface of the polymer active polymer layer to obtain the conventional optical biosensor. However, the method for manufacturing the conventional optical biosensor is dangerous in operation due to the strong acid solution or the strong alkali solution used, and a large amount of waste containing the strong acid solution or the strong alkali solution will also pollute the environment.

In addition, in the method for manufacturing the conventional optical biosensor, the negative charges on the surface of the silicon-containing substrate can also be formed by oxygen plasma. However, special instruments such as an oxygen plasma cleaner is needed to perform oxygen plasma treatment under specialized conditions such as high temperature and high pressure, greatly increasing the cost for manufacturing the conventional optical biosensor.

In light of this, it is necessary to improve the conventional method for manufacturing the biosensor.

### SUMMARY OF THE INVENTION

It is therefore an objective of the present invention to provide a method for manufacturing a biosensor in which a strong acid solution or a strong alkali solution is not required.

It is another objective of the present invention to provide a method for manufacturing a biosensor with lower manufacturing cost.

When the terms “front”, “rear”, “left”, “right”, “up”, “down”, “top”, “bottom”, “inner”, “outer”, “side”, and similar terms are used herein, it should be understood that these terms have reference only to the structure shown in the drawings as it would appear to a person viewing the drawings and are utilized only to facilitate describing the invention, rather than restricting the invention.

As used herein, the term “a” or “an” for describing the number of the elements and members of the present invention is used for convenience, provides the general meaning of the scope of the present invention, and should be interpreted to include one or at least one. Furthermore, unless explicitly indicated otherwise, the concept of a single component also includes the case of plural components.

One embodiment of the present invention discloses the method for manufacturing the biosensor. The method includes forming negative charges on at least one surface of a silicon-containing substrate by an ethanol solution. At least one active polymer layer with positive charges is formed on the at least one surface of the silicon-containing substrate. Each of the at least one active polymer layer has a coupling surface coupling to the silicon-containing substrate and an active surface opposite to the coupling surface. A plurality of capture biomolecule couples to the active surface of the at least one active polymer layer.

Accordingly, in the method for manufacturing the biosensor according to the present invention, by the use of the ethanol solution, the at least one surface of the silicon-containing substrate can be provided with negative charges without using the strong acid solution or the strong alkali solution. That is, it can not only improve the safety of the working environment, but also reduce the processing cost of the waste containing the strong acid solution and the strong alkali solution, and can prevent the discharge of the waste containing the strong acid solution and the strong alkali solution to organisms or buildings.

Moreover, in the method for manufacturing the biosensor according to the present invention, by the use of the ethanol solution, the at least one surface of the silicon-containing substrate can be provided with negative charges without using special instruments such as the oxygen plasma cleaner. The high temperature and high pressure environment required for oxygen plasma processing is also emitted; and therefore, the manufacturing cost of the biosensor can be reduced.

In preferred form shown, the negative charges on at least one surface of a silicon-containing substrate are formed by an aqueous ethanol solution with an ethanol concentration ranging from 60% to 99.8%. As such, by the use of the aqueous ethanol solution with a suitable ethanol concentration, the at least one surface of the silicon-containing substrate can be provided with a sufficient amount of negative charges, so that the at least one surface of the silicon-containing substrate can be stably couple to the at least one active polymer layer with the positive charges.

In preferred form shown, each of the plurality of capture biomolecules has negative charges, and the plurality of capture biomolecules electrostatic bonds to the active surface of the at least one active polymer layer. As such, compared with the use of the cross-linker, the method for manufacturing the biosensor according to the present invention has less complicate steps for coupling the plurality of capture biomolecules to the at least one active polymer layer.

In preferred form shown, the plurality of capture biomolecules couples to a covered area of the active surface of the at least one active polymer layer. The active surface of the at least one active polymer layer further includes an exposed area to which the plurality of capture biomolecules does not couple. Preferably, the method further includes covering the exposed area of the active surface of the at least one active polymer layer by a blocking layer. As such, by covering the exposed area of the active surface with the blocking layer, it is possible to prevent the impurities in a specimen from nonspecifically binding to the active surface, thereby improving the detection specificity of the biosensor.

In preferred form shown, each of the plurality of capture biomolecules couples to the active surface of the at least one active polymer layer via a noble metal nanoparticle. As an example, each of the plurality of noble metal nanoparticles has negative charges, each of the plurality of noble metal nanoparticles electrostatically bonds to the active surface of the at least one active polymer layer, and each of the plurality of capture biomolecules covalently bonds to the corresponding one of the plurality of noble metal nanoparticles. As such, with the existence of the noble metal nanoparticle, a larger steric hindrance can be formed, so that the plurality of capture biomolecules can be exposed more easily, and the detection sensitivity of the biosensor can be improved.

In preferred form shown, the plurality of noble metal nanoparticle couples to a covered area of the active surface of the at least one active polymer layer. The active surface of the at least one active polymer layer further includes an exposed area to which the plurality of noble metal nanoparticle does not couple. Preferably, the method further includes covering the exposed area of the active surface of the at least one active polymer layer by a blocking layer. As such, by covering the exposed area of the active surface with the blocking layer, it is possible to prevent the impurities in a specimen from nonspecifically binding to the active surface, thereby improving the detection specificity of the biosensor.

In preferred form shown, the active surface of the at least one active polymer layer has a functional group selected from a group consisting of an amino group and an ammonium group. As such, by the use of such functional group, the active surface of the at least one active polymer layer can have strong positive charges and can quickly couple to the capture biomolecules.

In preferred form shown, each of the at least one active polymer layer is formed by a polymer selected from a group consisting of polyethylenimine (such as a linear polyethylenimine or a branched polyethylenimine), poly(allylamine hydrochloride), poly(β-amino ester) (such as a linear poly(β-amino ester) or a branched poly(β-amino ester)), polydiallyldimethylammonium chloride and polyacrylamide. As such, due to the at least one active polymer layer is formed by such polymer, the active surface of the at least one active polymer layer can have strong positive charges and can quickly couple to the capture biomolecules.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

FIG. 1 depicts a cross sectional view of the biosensor manufactured by the method according to the first embodiment.

FIG. 2 depicts an enlarged view of A region of the biosensor of FIG. 1.

FIG. 3 depicts a cross sectional view of the biosensor of FIG. 2 after forming the blocking layer.

FIG. 4 depicts the spectrum ranging from 300 nm to 500 nm of the glass test piece of group A1 that is not pretreated by the aqueous ethanol solution, as well as the spectrum ranging from 300 nm to 500 nm of the glass test pieces of groups A2 to A4 that are treated by an aqueous branched PEI solution of 0.1 wt %, 1.0 wt % and 2.5 wt %, respectively, after pretreated by the aqueous ethanol solution in trial (A).

FIG. 5 depicts in trial (B), the calibration curve of the grayscale of the mixture to SARS-CoV-2-specific IgM antibody (●), as well as the calibration curve of the grayscale of the mixture to SARS-CoV-2-specific IgG antibody (▴).

FIG. 6 depicts in trial (C), the calibration curve of the absorbance of the mixture to the concentration of SARS-CoV-2-specific IgM antibody.

FIG. 7 depicts in trial (D), a bar chart illustrating the absorbance of SARS-CoV-2-specific IgM antibody and SARS-CoV-2-specific IgG antibody of the blood serum specimen from healthy objects (group D1), of SARS-CoV-2-specific IgM antibody and SARS-CoV-2-specific IgG antibody of the blood serum specimen from patients with other diseases (group D2), of SARS-CoV-2-specific IgM antibody and SARS-CoV-2-specific IgG antibody of the blood serum specimen in the early stage of SARS-CoV-2 virus infection (group D3), and of SARS-CoV-2-specific IgM antibody and SARS-CoV-2-specific IgG antibody of the blood serum specimen in the middle and late stage of SARS-CoV-2 virus infection (group D4).

FIG. 8 depicts a cross sectional view of the biosensor manufactured by the second embodiment.

FIG. 9 depicts a cross sectional view of the biosensor of FIG. 8 after forming the blocking layer.

FIG. 10 depicts a flow chart for using the biosensor manufactured by the second embodiment.

FIG. 11 depicts in trial (E), the spectrum ranging from 400 nm to 650 nm of the glass test piece of group E1 that the plurality of noble metal nanoparticles is absent, as well as the spectrum ranging from 400 nm to 650 nm of the glass test piece of group E2 that the plurality of noble metal nanoparticles couples to the active polymer layer.

FIG. 12 depicts in trial (F), the fluorescent image of the glass test piece of group F1 that the plurality of capture biomolecules is absent.

FIG. 13 depicts in trial (F), he fluorescent image of the glass test piece of group F2 that the plurality of capture biomolecules respectively couples to the plurality of noble metal nanoparticles.

FIG. 14 depicts in trial (G), a bar chart illustrating the surface roughness of the untreated glass test piece (group G1), of the glass test piece with the active polymer layer (group G2), of the glass test piece with the plurality of noble metal nanoparticles (group G3), of the glass test piece with the plurality of capture biomolecules (group G4), and of the glass test piece with the blocking layer (group G5).

FIG. 15 depicts in trial (H), the spectrum ranging from 350 nm to 550 nm of the urine specimen with different concentration of FXYD3 protein.

FIG. 16 depicts in trial (I), a bar chart illustrating FXYD3 protein level of the urine specimen from the healthy subject (group I1), of the urine specimen from patients with low-grade urothelial carcinoma of bladder (UCB) (group I2), of the urine specimen from patients with low-grade upper urinary tract urothelial carcinoma (UTUC) (group I3), of the urine specimen from patients with high-grade urothelial carcinoma of bladder (UCB) (group I4), and of the urine specimen from patients with high-grade upper urinary tract urothelial carcinoma (UTUC) (group I5).

### DETAILED DESCRIPTION OF THE INVENTION

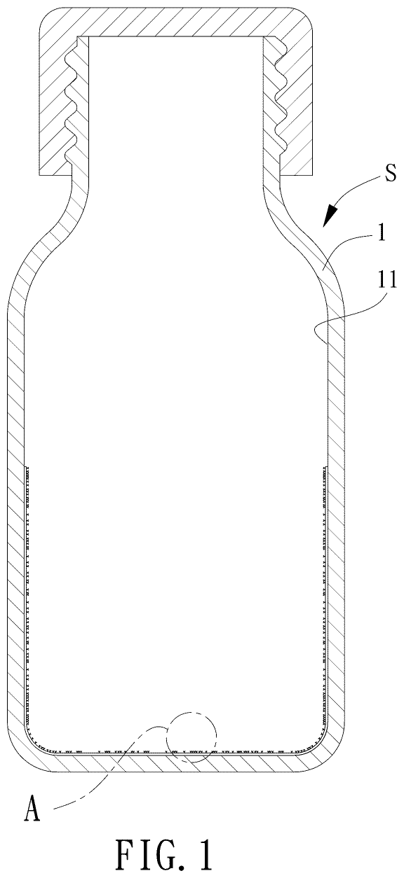

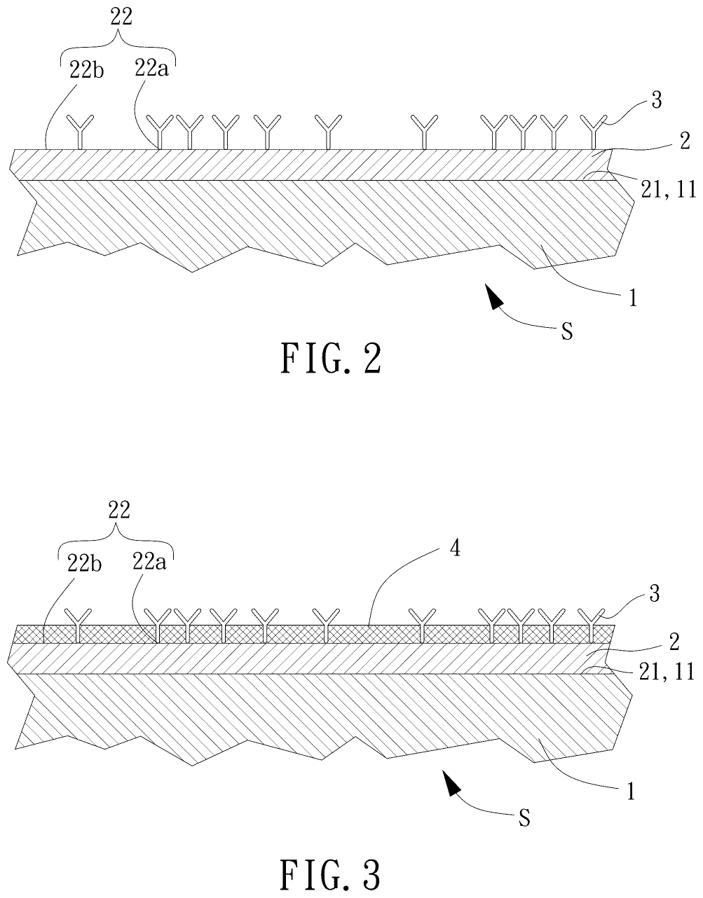

Referring to FIGS. 1 and 2, in a method for manufacturing a biosensor according to the present invention, a silicon-containing substrate 1 is first provided. An active polymer layer 2 is subsequentially formed on the silicon-containing substrate 1. Then, a plurality of capture biomolecules 3 couples to the active polymer layer 2.

Specifically, the silicon-containing substrate 1 can be a silicon dioxide (SiO₂)-based substrate in any three-dimensional form such as a chip, a bottle, etc., which can be appreciated by a person having ordinary skill in the art.

At least one surface 11 of the silicon-containing substrate 1 can form negative charges by an ethanol solution. As an example, the ethanol solution can be an aqueous ethanol solution with a concentration of ethanol ranging from 60% to 99.8%. It is worthy to note that impurities or grease on the at least one surface 11 of the silicon-containing substrate 1 might be effectively washed off if the concentration of ethanol of the aqueous ethanol solution is lower than 60%, and thus insufficient negative charges forms on the at least one surface 11 of the silicon-containing substrate 1 or uneven distribution of negative charges forms on the at least one surface 11 of the silicon-containing substrate 1. As a result, the active polymer layer 2 might not couples to the at least one surface 11 of the silicon-containing substrate 1 stably. A worker can use a glass vial as shown in FIG. 1 (with a volume of about 2 mL) as the silicon-containing substrate 1. The aqueous ethanol solution (with a volume of 1 mL) is added into the glass vial. After vortexing for about 10 minutes at room temperature (22° C. to 28° C.), the inner surface of the glass vial can form hydroxide ions (OH⁻ ions) with negative charges. Namely, the glass vial is the silicon-containing substrate 1, and the inner surface of the glass vial is the surface 11 of the silicon-containing substrate 1 that has negative charges.

In addition, a worker can use a glass chip as the silicon-containing substrate 1. After soaking the glass chip in the aqueous ethanol solution, the relative two surfaces of the glass chip can form hydroxide ions (OH⁻ ions) with negative charges. Namely, the glass chip is the silicon-containing substrate 1, and both the relative two surfaces of the glass chip are the surfaces 11 of the silicon-containing substrate 1 that have negative charges.

Moreover, before forming negative charges by the ethanol solution, the silicon-containing substrate 1 can be pretreated to remove dust, grease or impurities on the surface 11 of the silicon-containing substrate 1. As an example, a worker can wash the surface 11 of the silicon-containing substrate 1 by a tri(hydroxymethyl)aminomethane (Tris) buffer containing 0.10% polysorbate 20 (Tween 20) or wash the surface 11 of the silicon-containing substrate 1 by acetone or deionized water.

Referring to FIGS. 1 and 2, after obtaining the surface 11 having negative charges, a worker can form the active polymer layer 2 having positive charges on the surface 11. The active polymer layer 2 has a coupling surface 21 and an active surface 22 opposite to the coupling surface 21. The active polymer layer 2 can electrostatically bond to the surface 11 of the silicon-containing substrate 1 via the coupling surface 21. It is worthy to note that the active polymer layer 2 can preferably have a functional group having positive charges, and the functional group can be selected from a group consisting of an amine (—NH₂) group and an ammonium (—NH₄) group. Thus, the active polymer layer 2 can couple to the plurality of capture biomolecules 3 by the functional group. As an example, the active polymer layer 2 can be formed by a polymer, and the polymer can be selected from a group consisting of polyethylenimine (PEI) such as linear PEI or branched PEI, poly(allylamine hydrochloride) (PAH), poly(β-amino ester) (PAE) such as linear PAE or branched PAE, polydiallyldimethylammonium chloride (PDDA) and polyacrylamide.

In this embodiment, a worker can prepare an aqueous solution of branched PEI (0.1 wt %, Co #408727, purchased from Sigma-Aldrich). The aqueous solution of branched PEI (0.1 mL) can be added into the glass vial. After standing for 2 hours at room temperature, the active polymer layer 2 with positive charges (amine group) can be formed by branched PEI, and the active polymer layer 2 can electrostatically bond to the inner surface of the glass vial (that is, the surface 11 of the silicon-containing substrate 1) via the coupling surface 21. Branched PEI that is not electrostatically bond to the inner surface of the glass vial can be washed off by deionized water. Moreover, the glass vial is then heated to 80° C. for more than 15 minutes and gradually cool to room temperature to strengthen the coupling force between the active polymer layer 2 and the silicon-containing substrate 1.

Then, referring to FIGS. 1 and 2, the plurality of capture biomolecules 3 can couple to the active surface 22 of the active polymer layer 2 (that is, the surface that the active polymer layer 2 not couples to the silicon-containing substrate 1), and the active polymer layer 2 can be sandwiched between the plurality of capture biomolecules 3 and the silicon-containing substrate 1 to form a biosensor S. A worker can choose the capture biomolecule 3 according to a target biomolecule to be specifically detected by the biosensor S. As an example, the capture biomolecule 3 can be an antibody, an antigen, an enzyme, a substrate, an aptamer, etc., and thus the biosensor S can specifically detect the corresponding target biomolecule such as antigen, antibody, substrate, enzyme, nucleic acids and cell.

The plurality of capture biomolecules 3 can electrostatically bond to the active surface 22 of the active polymer layer 2 having positive charges. As an example, a worker can choose the capture biomolecules that originally have negative charges as the capture biomolecules 3. In another way, the pH value of the solution of the capture biomolecules 3 can be adjusted to be a pH value that is higher than the isoelectric point (pI) of the capture biomolecule 3, and thus the capture biomolecule 3 can have negative charges. Also, the capture biomolecule 3 can be modified by a thiol (—SH) group, and thus the capture biomolecule 3 can have negative charges. Accordingly, the capture biomolecule 3 having negative charges can electrostatically bond to the active surface 22 of the active polymer layer 2.

In this embodiment, SARS-CoV-2 nucleocapsid protein having negative charges is used as the capture biomolecule 3. The SARS-CoV-2 nucleocapsid protein can specifically bind to SARS-CoV-2-specific immunoglobulin M (IgM) antibody and can also specifically bind to SARS-CoV-2-specific immunoglobulin G (IgG) antibody. Namely, the biosensor S including the SARS-CoV-2 nucleocapsid protein can be applied to detect whether a specimen includes the SARS-CoV-2-specific IgM antibody and/or the SARS-CoV-2-specific IgG antibody. The SARS-CoV-2 nucleocapsid protein can be dissolved in a phosphate buffered saline (PBS) to form a solution of SARS-CoV-2 nucleocapsid protein (100 ng/mL). The solution of SARS-CoV-2 nucleocapsid protein (0.1 mL) is then added into the glass vial, and stand for 1 hour at room temperature. The SARS-CoV-2 nucleocapsid protein having negative charges can thus electrostatically bond to the active surface 22 of the active polymer layer 2 on the inner surface of the glass vial.

In addition, the plurality of capture biomolecules 3 may not form a layer of capture biomolecules 3 that totally cover the active surface 22. Namely, the active surface 22 has an area to which the plurality of capture biomolecules 3 does not couple. Thus, the active surface 22 can be divided into a covered area 22a to which the plurality of capture biomolecules 3 couples and an exposed area 22b to which the plurality of capture biomolecules 3 does not couple. Moreover, the specimen derived from organism usually includes large amount of impurities such as serum albumin, bilirubin, lipid, hemoglobin etc. In order to prevent the impurities from nonspecific binding to the exposed area 22b of the active polymer layer 2, affecting the detection result of the biosensor S, a blocking solution such as a bovine serum albumin (BSA) solution or a casein solution can be further added into the glass vial, forming a blocking layer 4 that covers the exposed area 22b of the active surface 22 of the active polymer layer 2. In this embodiment, the blocking solution is an aqueous BSA solution with a concentration of BSA being 2 wt %. The aqueous BSA solution (1 mL) is added into the glass vial, and the blocking layer 4 can be formed on the exposed area 22b of the active surface 22 of the active polymer layer 2 on the inner surface of the glass vial after standing for 1 hour at room temperature. After washing off the unbound BSA by the Tris buffer, the biosensor S shown in FIG. 3 can be obtained.

The biosensor S manufactured by the method according to the first embodiment can be used as follows:

Preparation of the probe solution: The probe solution includes the horseradish peroxidase (HRP)-tagged anti-human IgM secondary antibody (250 ng/mL) and/or the HRP-tagged anti-human IgG secondary antibody (250 ng/mL), dissolved in the Tris buffer including 0.001% polysorbate 20 (Tween 20).

Preparation of the chromogen solution: The chromogen solution includes 3,3′,5,5′-tetramethylbenzidine (TMB) (0.5 mg/mL) and hydrogen peroxide (H₂O₂) (0.5%), dissolved in the aqueous sodium acetate (NaOAc) (0.1M, pH 5.5).

Preparation of the terminating reagent: The terminating reagent includes the aqueous hydrochloric acid (HCl) solution (1 M).

Preparation of the specimen: In order to determine whether a suspected patient is infected by a SARS-CoV-2 virus using the biosensor S, the specimen can be a whole blood specimen (such as the venous whole blood specimen or the finger-prick whole blood specimen), a blood serum specimen, a blood plasma specimen, a urine specimen or a saliva specimen from the suspected patient. In this embodiment, the finger-prick whole blood specimen is used for convenience of Point of Care Testing.

The use process of the biosensor S is as follows:

The probe solution (1 mL) is added into the glass vial. The finger-prick whole blood specimen (5 μL) is then added into the glass vial, followed by mixing uniformly. After standing for 15 minutes at room temperature, the HRP-tagged anti-human IgM secondary antibody and/or the HRP-tagged anti-human IgG secondary antibody in the probe solution can specifically bind to the human anti-SARS-CoV-2 IgM antibody and/or the human anti-SARS-CoV-2 IgG antibody of the finger-prick whole blood specimen, and thus can specifically bind to the capture biomolecules 3 (SARS-CoV-2 nucleocapsid protein) on the inner surface of the glass vial.

After washing the glass vial, the chromogen solution (0.5 mL) is added into the glass vial. After standing for 2 minutes, the color of the mixture in the glass vial changes. As this time, under the action of HRP, the mixture in the glass vial will gradually turn dark blue.

Finally, the terminating reagent is added into the mixture in dark blue. At this time, under the action of the terminating reagent, the mixture in the glass vial will turn yellow from dark blue. Therefore, a worker can observe the color change of the mixture with the naked eye. Alternatively, a worker can observe the absorbance change at a specific wavelength (such as at 450 nm) of the mixture with a spectrometer.

In the situation that in the use of the biosensor S manufactured by the method according to the first embodiment, if the suspected patient is infected by SARS-CoV-2 virus, the capture biomolecules 3 (SARS-CoV-2 nucleocapsid protein) can capture human anti-SARS-CoV-2 IgM antibody and/or human anti-SARS-CoV-2 IgG antibody in the finger-prick whole blood specimen from the suspected patient, and bind to the HRP-tagged anti-human IgM secondary antibody and/or the HRP-tagged anti-human IgG secondary antibody in the probe solution. Therefore, the chromogen solution added into the glass vial will change its color under the action of HRP. Namely, the dark blue color of the mixture in the glass vial indicates that the suspected patient has been infected by SARS-CoV-2 virus.

In order to evaluate the specificity of the biosensor S manufactured by the method according to the first embodiment to SARS-CoV-2 virus, the following trials are carried out.

### Trial (A).

In trial (A), as shown in TABLE 1, a glass test piece is first soaked in an aqueous ethanol solution (95%). The glass test piece is then soaked in an aqueous branched PEI solution (0.1 wt %) for 1 hour at room temperature. After washing unbound branched PEI, the glass test piece is soaked in an aqueous 2,4,6-trinitrobenzenesulfonic acid (TNBS) solution. TNBS will react with the amine group on the glass test piece, forming a chromophore having largest absorbance at 340 nm. The absorbance from 300 nm to 500 nm of the glass test piece (group A2) is finally detected.

As shown in TABLE 1, the aqueous branched PEI solution (1.0 wt %) and the aqueous branched PEI solution (2.5 wt %) are used to replace the aqueous branched PEI solution (0.1 wt %) to obtain the glass test pieces of groups A3 and A4, respectively.

Moreover, the glass test piece without soaking in the aqueous ethanol solution is soaked in the aqueous branched PEI solution (0.1 wt %) to obtain the glass test piece of group A1.

TABLE 1

| | | | Active |
|---|---|---|---|
| | Silicon-containing | Ethanol | polymer |
| Group | substrate 1 | treatment | layer 2 |
| A1 | Glass chip | − | Branched PEI |
| | | | (0.1 wt %) |
| A2 | Glass chip | + | Branched PEI |
| | | | (0.1 wt %) |
| A3 | Glass chip | + | Branched PEI |
| | | | (1.0 wt %) |
| A4 | Glass chip | + | Branched PEI |
| | | | (2.5 wt %) |

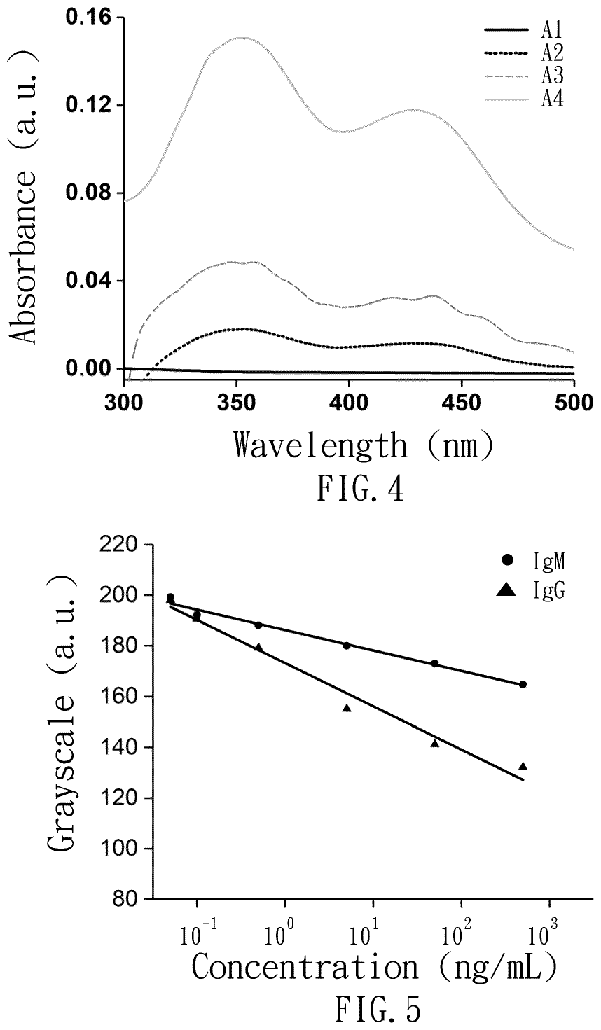

Referring to FIG. 4, due to the insufficient hydroxide ion (OH⁻) having negative charges on the surface of the glass test piece of group A1, the active polymer layer 2 cannot electrostatically bond to the surface of the glass test piece of group A1 that is not treated by the aqueous ethanol solution; and thus, a characteristic peak cannot be observed at 340 nm. Moreover, the characteristic peak at 340 nm can be observed in the glass test pieces of groups A2-A4 that are treated by the aqueous ethanol solution, indicating by the treatment of the aqueous branched PEI solution (0.1-2.5 wt %), amine groups having positive charges can be formed on the surface of the glass test pieces. That is, the active polymer layer 2 is formed on the surface of the glass test pieces.

### Trial (B).

In trial (B), a standard of human anti-SARS-CoV-2 IgM antibody and a standard of human anti-SARS-CoV-2 IgG antibody are used. The standards are diluted to 10⁻¹, 10⁰, 10¹, 10² and 10³ ng/mL, and are detected according to the abovementioned method. Finally, a smartphone (iPhone 7 plus) is used to take a photograph, and the grayscale of the mixture is calculated according the following equation, where R, G and B are the red color, green color and blue color values from the photograph taken by the smartphone, respectively.

$$grayscale = 0.299 \times R + 0.587 \times G + 0.114 \times B \qquad Equation ( 1 )$$

Referring to FIG. 5, by the use of both human anti-SARS-CoV-2 IgM antibody and human anti-SARS-CoV-2 IgG antibody, the linear calibration curve can be obtained, indicating the detection of the finger-prick whole blood specimen using the biosensor S manufactured by the method according to the first embodiment has great linearity.

### Trial (C).

In trial (C), a standard of human anti-SARS-CoV-2 IgM antibody and a standard of human anti-SARS-CoV-2 IgG antibody are used. The standards are diluted to 10^{1.0}, 10^{1.5}, 10^{2.0}, 10^{2.5}, 10³, 10^{3.5}, 10^{4.0} and 10^{4.5} pg/mL, and are detected according to the abovementioned method. Finally, a spectrometer (SpectraMax M2) is used to measure the absorbance at 450 nm.

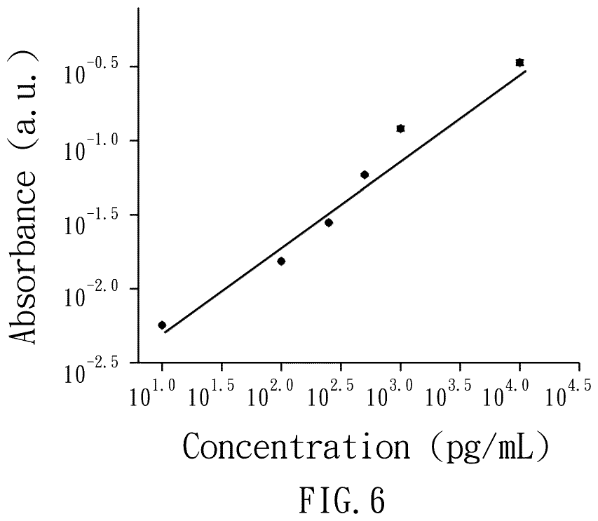

Referring to FIG. 6, the regression equation of the calibration curve is as follows, and the coefficient of determination (R²) of the regression equation is 0.98838.

$$y = - 2.69779 + 0.44698 x \qquad Equation ( 2 )$$

### Trial (D).

In trial (D), the blood serum specimen from healthy objects are used as group D1 (10 cases), the blood serum specimen from patients with other diseases such as influenza A/B, pneumonia, tuberculosis, lung cancer or liver cancer (the patients have fever, cough, sore throat, runny nose etc.) are used as group D2 (109 cases) and the blood serum specimen from patients infected by SARS-CoV-2 virus are used as groups D3-D4 (29 cases). The blood serum specimen of groups D3-D4 are divided into specimen in the early stage of infection (group D3, 8 cases) and specimen in the middle and late stages of infection (group D4, 21 cases) according the detected IgM and IgG level, as well as the detected SARS-CoV-2 virus level. The blood serum specimen of groups D1-D4 are detected according to the abovementioned method. Finally, a spectrometer (SpectraMax M2) is used to measure the absorbance at 450 nm.

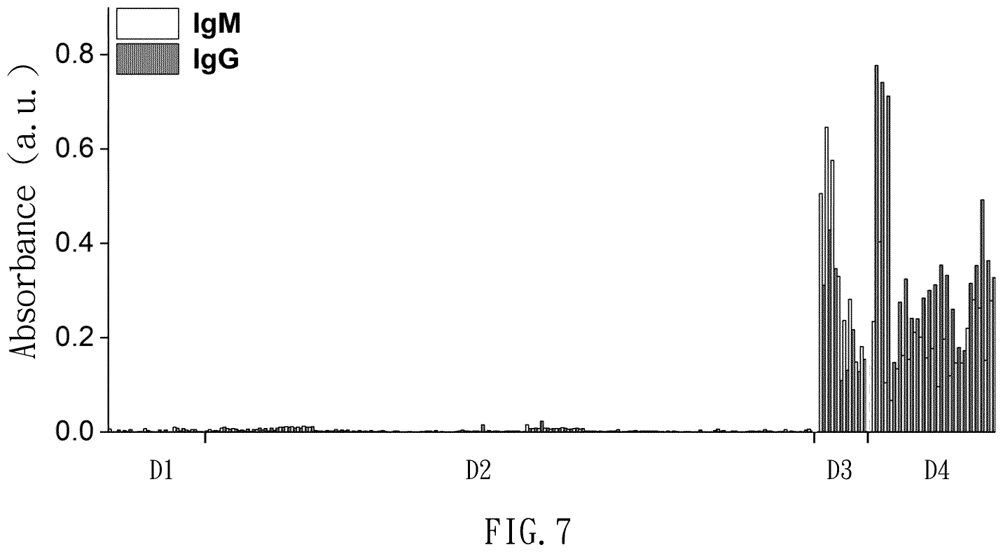

Referring to FIG. 7, neither SARS-CoV-2-specific IgM antibody nor SARS-CoV-2-specific IgG antibody can be detected in the blood serum specimen from healthy objects (group D1) or the blood serum specimen from patients with other diseases (group D2). Moreover, both SARS-CoV-2-specific IgM antibody and SARS-CoV-2-specific IgG antibody can be detected in the blood serum specimen from patients infected by SARS-CoV-2 virus (groups D3-D4). In the blood serum specimen in the early stage of infection (group D3), the level of SARS-CoV-2-specific IgM antibody is higher than the level of SARS-CoV-2-specific IgG antibody, and in the blood serum specimen in the middle and late stages of infection (group D4), the level of SARS-CoV-2-specific IgG antibody is higher than the level of SARS-CoV-2-specific IgM antibody.

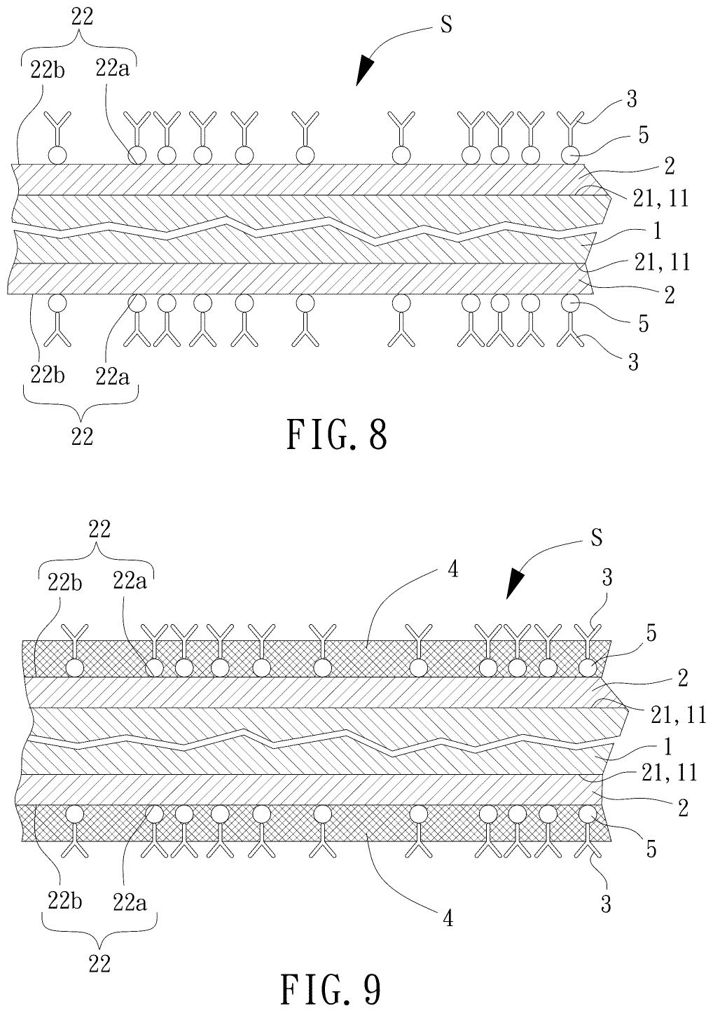

Based on the same technical concept, referring to FIG. 8, in the method for manufacturing the biosensor S according to the second embodiment, the active polymer layer 2 is also formed on the silicon-containing substrate 1. The plurality of capture biomolecules 3 then couples to the active polymer layer 2.

In this embodiment, a glass chip is used as the silicon-containing substrate. The glass chip is soaked in the aqueous ethanol solution with a concentration of ethanol ranging from 60% to 99.8% for 1 hour, forming hydroxide ions (OH⁻ ions) on the relative two surfaces of the glass chip. In other words, in this embodiment, the relative two surfaces of the glass chip that forms the hydroxide ions with negative charges correspond to two surfaces 11 of the silicon-containing substrate 1. Subsequently, the glass chip is soaked in the aqueous branched polyethylenimine (PEI) solution (0.1 wt %) at room temperature for 2 hours, forming two active polymer layers 2 with positive charges by branched polyethylenimine (PEI). Each of the two active polymer layers 2 electrostatically bond to the relative two surfaces of the glass chip (that is, the two surfaces 11 of the silicon-containing substrate 1) via the coupling surface 21.

It is worthy to note that in the method for manufacturing the biosensor S according to the second embodiment, each of the plurality of capture biomolecules 3 indirectly couples to the active surface 22 of the active polymer layer 2 via the noble metal nanoparticles 5.

Specifically, the noble metal nanoparticles 5 with negative charges can be used, and thus the noble metal nanoparticles can electrostatically bond to the active surface 22 of the active polymer layer 2 with positive charges. Moreover, by the covalent bond forming between the noble metal nanoparticles 5 and the capture biomolecules 3, the plurality of capture biomolecules 3 can couple to the active surface 22 of the active polymer layer 2 via noble metal nanoparticles 5. As an example, the noble metal nanoparticles 5 can be selected from the group consisting of a gold (Au) nanoparticle, a platinum (Pt) nanoparticle, silver (Ag) nanoparticle and a palladium (Pd) nanoparticle.

Moreover, the noble metal nanoparticles 5 can electrostatically bond to the active surface 22 of the active polymer layer 2, followed by covalently bonding to the capture biomolecules 3. Alternatively, the noble metal nanoparticles 5 can form a complex with the capture biomolecules 3 via covalent bonds, followed by electrostatically bonding to the active surface 22 of the active polymer layer 2 via the noble metal nanoparticles 5 of the complex; and thus the capture biomolecules 3 can indirectly couple to the active surface 22 of the active polymer layer 2 by the noble metal nanoparticles 5, which can be appreciated by a person having ordinary skill in the art.

In this embodiment, an aqueous sodium citrate (Na₃C₆H₅O₇) solution (10 mL, 38.8 M) is added into a boiling aqueous chloroauric acid (H[AuCl₄]) solution (about 100° C.). After a color of a mixture including the aqueous sodium citrate solution and the boiling aqueous chloroauric acid solution turns from light yellow to wine, the mixture is slowly cooled to room temperature. A plurality of gold nanoparticles with a particle size ranging from 10 nm to 50 nm can be formed. Finally, the plurality of gold nanoparticles can be washed, and can be resuspended in deionized water to obtain a suspension of gold nanoparticles.

Subsequently, the glass chip is soaked in the suspension of gold nanoparticles (0.5 mL) at room temperature, thus the plurality of gold nanoparticles can electrostatically bond to the active surface 22 of the active polymer layer 2. After washing by deionized water, the glass chip is soaked in an antibody solution (0.5 mL, with 500 ng/mL of thiolated anti-FXYD3 rabbit polyclonal antibody, dissolved in phosphate buffered saline (PBS)) at room temperature for 2 hours, and thus the anti-FXYD3 rabbit polyclonal antibody can form covalent bonds with the plurality of gold nanoparticles, and can couple to the active surface 22 of the active polymer layer 2 via the plurality of gold nanoparticles. Accordingly, the biosensor S shown in FIG. 8 can be obtained.

In addition, the glass chip can also be treated by the blocking solution, forming the blocking layer 4 that covers the exposed area 22b of the active surface 22 (that is, the area where the noble metal nanoparticles 5 are not bound). In this embodiment, the glass chip is soaked in the aqueous BSA solution (with 2 wt % of BSA) at room temperature for 1 hour to form the blocking layer 4 on the exposed area 22b of the active surface 22 of the active polymer layer 2 on the relative two surfaces of the glass chip. After washing with the Tris buffer, the biosensor S shown in FIG. 9 can be obtained.

The biosensor S manufactured by the method according to the second embodiment can be used as follows:

Preparation of the probe solution: The thiolated anti-FXYD3 rabbit polyclonal antibody (10 ng/μL, 10 μL) and horseradish peroxidase (HRP, 20 mg/mL, 10 μL) are added to the suspension of gold nanoparticles at room temperature in the dark for 2 hours. After the thiolated anti-FXYD3 rabbit polyclonal antibody and horseradish peroxidase (HRP) couple to the gold nanoparticle, the mixture is centrifugated at 12,000 rpm for 10 minutes, and the supernatant is discarded. The aqueous BSA solution (200 μL) including 2 wt % of BSA is added for 30 minutes. The mixed solution is centrifugated (12,000 rpm, 10 minutes) and the supernatant is discarded again. Finally, after fully washing by the Tris buffer (including 0.10% Tween 20, 200 μL) and discarding the supernatant, the obtained probe particle is resuspended in phosphate buffered saline (PBS, 200 μL) to form the probe solution.

Preparation of the washing solution: The washing solution includes the Tris buffer with 0.001% of Tween 20.

Preparation of the chromogen solution: The chromogen solution includes 3,3′,5,5′-tetramethylbenzidine (TMB) (0.5 mg/mL) and hydrogen peroxide (H₂O₂) (0.5%), dissolved in the aqueous sodium acetate (NaOAc) (0.1M, pH 5.5).

Preparation of the terminating reagent: The terminating reagent includes the aqueous HCl solution (1 M).

Preparation of the specimen: In order to determine whether a suspected patient suffers from urothelial carcinoma (UC) using the biosensor S, the specimen can be a whole blood specimen, a blood serum specimen, a blood plasma specimen or a urine specimen from the suspected patient. In this embodiment, for convenience of Point of Care Testing of urothelial carcinoma (UC), a clean-catch urine sample is used. The urine specimen is centrifugated at 5,000 rpm for 10 minutes at 4° C. The obtained precipitate is treated by a protein extractant (PROPREP protein extraction solution, purchased from iNtRON Biotechnology, Inc., Cat. No. 17081) for 15 minutes. After lysing the cells in the urine specimen, the sample is then centrifugated at 13,000 rpm for 10 minutes at 4° C. The obtained supernatant is stored at −80° C. and can be used as the clean-catch urine sample.

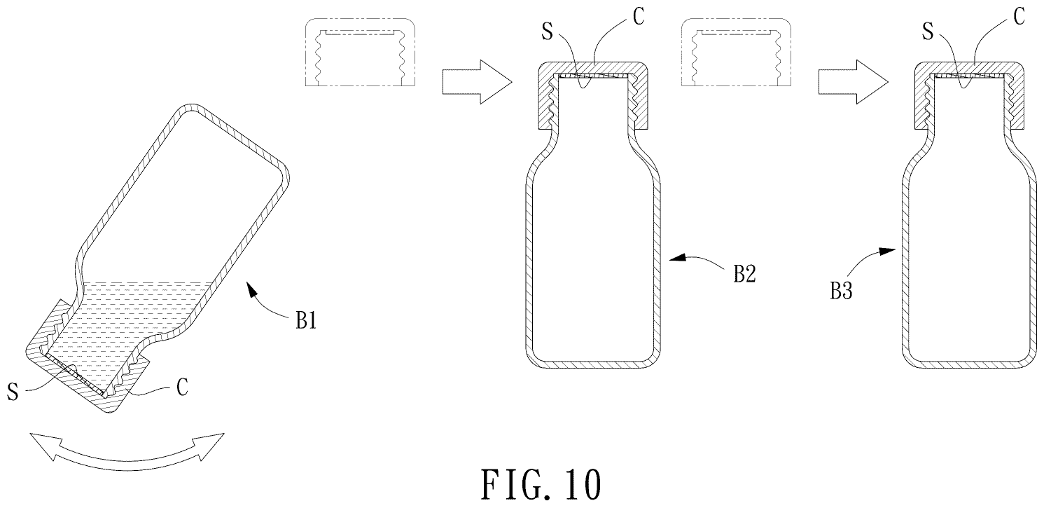

Referring to FIG. 10, the use process of the biosensor S is as follows:

The biosensor S shown in FIG. 9 is sticked on the inside of a cap C. Thus, when the cap C couples to a bottle, the capture biomolecules 3 of the biosensor S faces the bottle.

The probe solution (0.15 mL) and the urine specimen (0.05 mL) are sequentially added into a plastic bottle B1, and the cap C is used to close the plastic bottle B1. After shaking, the mixture in the plastic bottle B1 stands at room temperature for 15 minutes; and thus, the probe particle in the probe solution can specifically bind to the FVYD3 protein in the urine specimen, and then specifically bind to the capture biomolecule 3 (anti-FXYD3 rabbit polyclonal antibody) on the inner surface of the glass vial.

After open the plastic bottle B1, the cap C is changed to another plastic bottle B2 with the washing solution (1 mL) inside. The plastic bottle B2 is shaken to wash the biosensor S inside the cap C.

Again, the cap C is changed to a glass bottle B3 with the chromogen solution (0.2 mL) inside. After shaking and standing for 2 minutes, the color change of the mixture in the glass bottle B3 is observed. Under the action of horseradish peroxidase (HRP), the mixture in the glass bottle B3 will gradually turn into dark blue.

Finally, the terminating reagent is added to the mixture in dark blue. Under the action of the terminating reagent, the mixture in dark blue will turn into yellow. As such, a worker can observe the color change of the mixture with the naked eye. Alternatively, a worker can use a spectrometer to measure the absorbance change at a specific wavelength (such as 450 nm).

In the situation that in the use of the biosensor S manufactured by the method according to the second embodiment, if the suspected patient suffers urothelial carcinoma (UC), the capture biomolecules 3 (rabbit anti-FXYD3 polyclonal antibody) can capture FXYD3 protein in the urine specimen from the suspected patient. Therefore, the chromogen solution added into the glass vial will change its color under the action of HRP. Namely, the dark blue color of the mixture in the glass bottle B3 indicates the suspected patient has suffered from urothelial carcinoma (UC).

In order to evaluate the specificity of the biosensor S manufactured by the method according to the second embodiment to the biomarker (FXYD3 protein) of urothelial carcinoma (UC), the following trials are carried out.

### Trial (E).

Referring to TABLE 2, the active polymer layer 2 formed by branched polyethylenimine (PEI) is formed on the silicon-containing substrate 1 (glass chip) to obtain the glass test piece of group E1. Moreover, to obtain the glass test piece of group E2, after forming the active polymer layer 2 formed by branched polyethylenimine (PEI) on the silicon-containing substrate 1 (glass chip), the noble metal nanoparticles 5 (the gold nanoparticles) further electrostatically bond to the active surface 22 of the active polymer layer 2.

TABLE 2

| | Silicon-containing | Active polymer | Noble metal |
|---|---|---|---|
| Group | substrate 1 | layer 2 | nanoparticles 5 |
| E1 | Glass chip | Branched | — |
| | | polyethylenimine | |
| | | (PEI) | |
| E2 | Glass chip | Branched | Gold |
| | | polyethylenimine | nanoparticle |
| | | (PEI) | |

Then, the gold nanoparticles have the characteristic absorption peak at 520 nm due to the localized surface plasmon resonance (LSPR) characteristics (referring to Huang et al., 2020, Rapid Detection of IgM Antibodies against the SARS-CoV-2 Virus via Colloidal Gold Nanoparticle-Based Lateral-Flow Assay). Therefore, the absorbance at a wavelength ranging from 400 nm to 650 nm of the glass test pieces of groups E1 and E2 is detected.

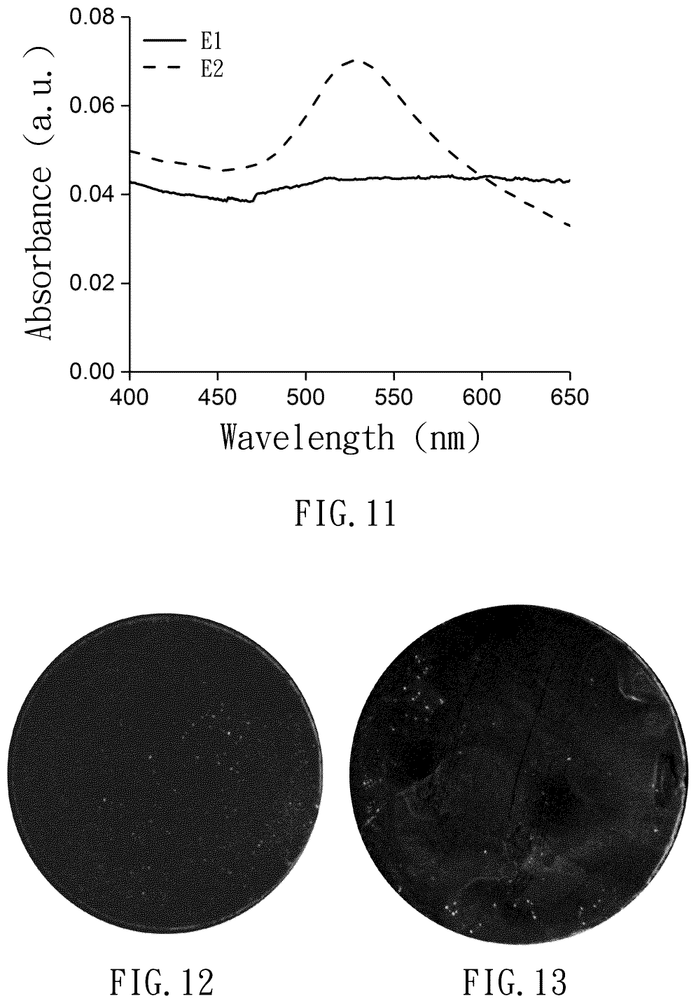

Referring to FIG. 11, the glass test piece of group E2 has a peak at 520 nm, indicating the noble metal nanoparticles 5 (that is, the gold nanoparticles) electrostatically bond to the active surface 22 of the active polymer layer 2.

### Trial (F).

Referring to TABLE 3, to obtain the glass test piece of group F1, the active polymer layer 2 formed by branched polyethylenimine (PEI) is first formed on the silicon-containing substrate 1 (glass chip). After electrostatically bonding the noble metal nanoparticles 5 (the gold nanoparticles) to the active surface 22 of the active polymer layer 2, the blocking layer 4 is formed by the blocking solution (the aqueous bovine serum albumin (BSA)). Moreover, to obtain the glass test piece of group F2, after electrostatically bonding the noble metal nanoparticles 5 (the gold nanoparticles) to the active surface 22 of the active polymer layer 2, the capture biomolecules 3 (anti-FXYD3 rabbit polyclonal antibody) are first covalently bond to the noble metal nanoparticles 5, and the blocking layer 4 is formed by the blocking solution (the aqueous bovine serum albumin (BSA).

TABLE 3

| | Silicon- | Active | Noble metal | Capture | |
|---|---|---|---|---|---|
| | containing | polymer | nanoparticles | biomolecules | Blocking |
| Group | substrate 1 | layer 2 | 5 | 3 | layer 4 |
| F1 | Glass chip | Branched | Gold | — | Bovine |
| | | poly- | nanoparticle | | serum |
| | | ethylenimine | | | albumin |
| | | (PEI) | | | (BSA) |
| F2 | Glass chip | Branched | Gold | Anti-FXYD3 | Bovine |
| | | poly- | nanoparticle | rabbit | serum |
| | | ethylenimine | | polyclonal | albumin |
| | | (PEI) | | antibody | (BSA) |

Then, the glass test pieces of groups F1 and F2 are treated with goat anti-rabbit iFluor 488 secondary antibody to label the capture biomolecules 3 in green fluorescent tag. The fluorescent image of the glass test pieces of groups F1 and F2 are taken Finally.

Referring to FIG. 12, almost no green, fluorescent signal can be observed in the fluorescent image of the glass test piece of group F1, indicating the biosensor B has good anti-interference ability to reduce non-specific molecular binding. Moreover, referring to FIG. 13, green fluorescent signal is evenly present in the fluorescent image of the glass test piece of group F2, indicating the capture biomolecules 3 (anti-FXYD3 rabbit polyclonal antibody) are evenly distributed on the glass test piece of group F2 by the noble metal nanoparticles 5.

### Trial (G).

In trial (G), referring TABLE 4, the untreated silicon-containing substrate 1 (glass chip) is used as the glass test piece of group G1. The active polymer layer 2 formed by branched polyethylenimine (PEI) is formed on the silicon-containing substrate 1 (glass chip) to obtain the glass test piece of group G2. To obtain the glass test piece of group G3, after forming the active polymer layer 2 formed by branched polyethylenimine (PEI) on the silicon-containing substrate 1 (glass chip), the noble metal nanoparticles 5 (the gold nanoparticles) further electrostatically bond to the active surface 22 of the active polymer layer 2. To obtain the glass test piece of group G4, after electrostatically bonding the noble metal nanoparticles 5 (the gold nanoparticles) to the active surface 22 of the active polymer layer 2, the capture biomolecules 3 (anti-FXYD3 rabbit polyclonal antibody) covalently bond to the noble metal nanoparticles 5 (the gold nanoparticles). Finally, to obtain the glass test piece of group G5, after covalently bonding the capture biomolecules 3 (anti-FXYD3 rabbit polyclonal antibody) to the noble metal nanoparticles 5 (the gold nanoparticles), the blocking layer 4 is formed by the blocking solution (the aqueous bovine serum albumin (BSA).

TABLE 4

| | Silicon- | Active | Noble metal | Capture | |
|---|---|---|---|---|---|
| | containing | polymer | nanoparticles | biomolecules | Blocking |
| Group | substrate 1 | layer 2 | 5 | 3 | layer 4 |
| G1 | Glass chip | — | — | — | — |
| G2 | Glass chip | Branched | — | — | — |
| | | poly- | | | |
| | | ethylenimine | | | |
| | | (PEI) | | | |
| G3 | Glass chip | Branched | Gold | — | — |
| | | poly- | nanoparticle | | |
| | | ethylenimine | | | |
| | | (PEI) | | | |
| G4 | Glass chip | Branched | Gold | Anti-FXYD3 | — |
| | | poly- | nanoparticle | rabbit | |
| | | ethylenimine | | polyclonal | |
| | | (PEI) | | antibody | |
| G5 | Glass chip | Branched | Gold | Anti-FXYD3 | Bovine |
| | | poly- | nanoparticle | rabbit | serum |
| | | ethylenimine | | polyclonal | albumin |
| | | (PEI) | | antibody | (BSA) |

Then, the surface roughness of the glass test pieces of groups G1 to G5 is observed by atomic force microscopy (AFM), respectively.

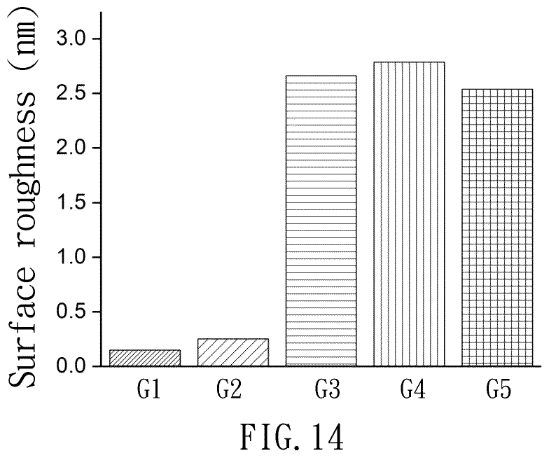

Referring to FIG. 14, the glass test piece of group G1 has the roughness average (Ra) of 0.149 nm, the glass test piece of group G2 has the roughness average (Ra) of 0.252 nm the glass test piece of group G3 has the roughness average (Ra) of 2.662 nm the glass test piece of group G4 has the roughness average (Ra) of 2.786 nm, and the glass test piece of group G5 has the roughness average (Ra) of 2.539 nm. That is, the surface roughness of the glass test piece increases due to the presence of the noble metal nanoparticles 5 and the capture biomolecules 3, while the surface roughness of the glass test piece decreases due to the formation of the blocking layer 4.

### Trial (H).

In trial (H), a standard sample with FXYD3 protein (that is, the biomarker of urothelial carcinoma (UC)) is used. The standard sample is added to a urine specimen from a healthy object in which FXYD3 protein is absent, forming the standard sample in the concentration of 1, 2.5, 10, 100, 500 and 1,000 pg/mL, respectively. After the detection using the biosensor B, the absorbance at 450 nm is measured using the spectrometer, and linear regression analysis is carried out.

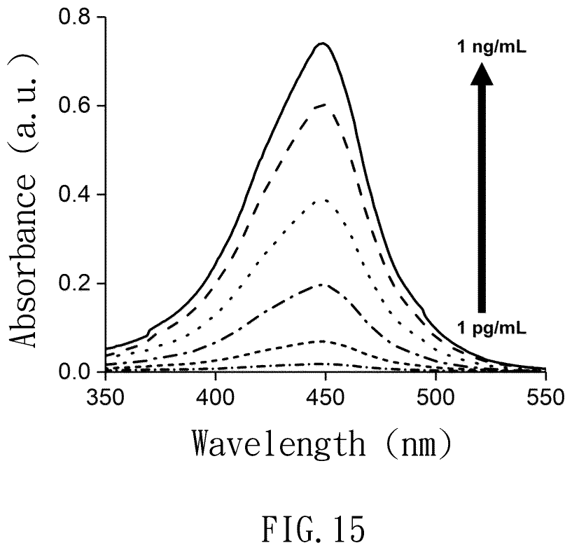

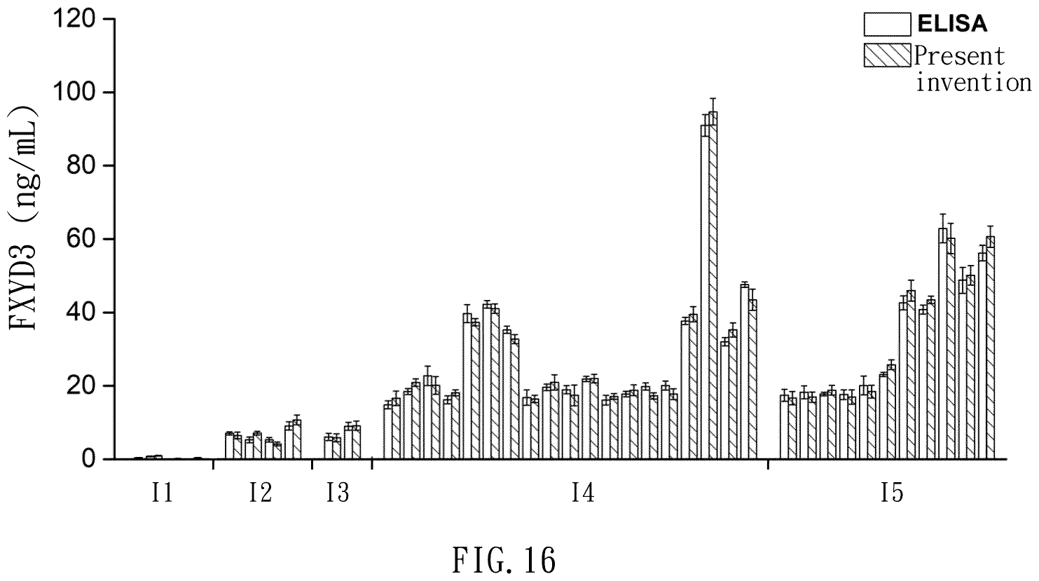

Referring to FIG. 15, as the concentration of FXYD3 protein in the urine specimen increases, the measured absorbance at 450 nm also increases. Moreover, the regression equation of the calibration curve is shown as equation 3(3), and the coefficient of determination (R²) and the regression equation is 0.9960.

$$y = - 0.01265 + 0.24182 x \qquad Equation ( 3 )$$

### Trial (I).

In trial (I), the patients who are diagnosed with urothelial carcinoma (UC) by clinical cystoscopy followed by biopsy. The urine specimens from low-grade urothelial carcinomas (UCs) (6 cases; 4 patients with urothelial carcinoma of bladder (UCB) (group I2), and 2 patients with upper urinary tract urothelial carcinoma (UTUC) (group I3)) and from high-grade urothelial carcinomas (UCs) (30 cases; 19 patients with urothelial carcinoma of bladder (UCB) (group I4), and 11 patients with upper urinary tract urothelial carcinoma (UTUC) (group I5)) are collected before standard clinical practice. Moreover, the urine specimens from healthy subjects (4 cases) are used as group I1.

After the detection using the biosensor B, the absorbance at 450 nm is measured using the spectrometer, and the concentration of FXYD3 protein in the respective urine specimen is calculated. Moreover, the absorbance at 450 nm measured using enzyme-linked immunosorbent assay (ELISA), and the corresponding concentration of FXYD3 protein are used as the control.

Referring to FIG. 16, the concentration of FXYD3 protein calculated according to the measured absorbance at 450 nm using the biosensor B in the urine specimen from healthy subjects (group I1), as well as in the urine specimen from the patients with urothelial carcinoma (UC) (groups 12 to 17) is similar to the concentration of FXYD3 protein calculated according to the measured absorbance at 450 nm using enzyme-linked immunosorbent assay (ELISA) from respective urine specimen.

According to the above data, a person having ordinary skill can understand that the biosensor S manufactured by the method according to the present invention has a preferable sensitivity. That is, the target biomolecules (SARS-CoV-2-specific immunoglobulin M (IgM) antibody, SARS-CoV-2-specific immunoglobulin G (IgG) antibody or FXYD3 protein) can be detected in the finger-prick whole blood specimen (or the urine specimen) in a volume of merely 5 to 50 μL. Moreover, compared to the conventional quantitative real-time polymerase chain reaction, the method using the biosensor S manufactured by the method according to the present invention is easier to carried out, and the time cost is also decreased. More importantly, the operator is not exposed to the risk of infection. In addition, compared to the conventional enzyme-linked immunosorbent assay (ELISA), the method using the biosensor S manufactured by the method according to the present invention is cheaper, and the time cost is significantly reduced to less than 15 minutes. Besides, the detection result can be distinguished by naked eye.

Accordingly, in the method for manufacturing the biosensor according to the present invention, by the use of the ethanol solution, the at least one surface of the silicon-containing substrate can be provided with negative charges without using the strong acid solution or the strong alkali solution. That is, it can not only improve the safety of the working environment, but also reduce the processing cost of the waste containing the strong acid solution and the strong alkali solution, and can prevent the discharge of the waste containing the strong acid solution and the strong alkali solution to organisms or buildings.

Moreover, in the method for manufacturing the biosensor according to the present invention, by the use of the ethanol solution, the at least one surface of the silicon-containing substrate can be provided with negative charges without using special instruments such as the oxygen plasma cleaner. The high temperature and high pressure environment required for oxygen plasma processing is also emitted; and therefore, the manufacturing cost of the biosensor can be reduced.

Although the invention has been described in detail with reference to its presently preferable embodiment, it will be understood by one of ordinary skill in the art that various modifications can be made without departing from the spirit and the scope of the invention, as set forth in the appended claims.

What is claimed is:

1. A method for manufacturing a biosensor, comprising:
forming negative charges on at least one surface of a silicon-containing substrate by an ethanol solution;
forming at least one active polymer layer having positive charges on the at least one surface of the silicon-containing substrate, wherein each of the at least one active polymer layer has a coupling surface and an active surface opposite to the coupling surface, and wherein the at least one active polymer layer couples to the silicon-containing substrate by the coupling surface; and
coupling the plurality of capture biomolecules to the active surface of the at least one active polymer layer.

2. The method for manufacturing the biosensor as claimed in claim 1, wherein the negative charges on the at least one surface of the silicon-containing substrate is formed by an aqueous ethanol solution with a concentration of ethanol ranging from 60% to 99.8%.

3. The method for manufacturing the biosensor as claimed in claim 1, wherein each of the plurality of capture biomolecules has negative charges, and wherein the plurality of capture biomolecules electrostatically bonds to the active surface of the at least one active polymer layer.

4. The method for manufacturing the biosensor as claimed in claim 3, wherein the plurality of capture biomolecules couples to a covered area of the active surface of the at least one active polymer layer, and wherein the active surface of the at least one active polymer layer further comprises an exposed area to which the plurality of capture biomolecules does not couple.

5. The method for manufacturing the biosensor as claimed in claim 4, further comprising covering the exposed area of the active surface of the at least one active polymer layer by a blocking layer.

6. The method for manufacturing the biosensor as claimed in claim 1, wherein each of the plurality of capture biomolecules couples to the active surface of the at least one active polymer layer via one corresponding one of plurality of noble metal nanoparticles.

7. The method for manufacturing the biosensor as claimed in claim 6, wherein each of the plurality of noble metal nanoparticles has negative charges, and wherein the plurality of noble metal nanoparticles electrostatically bonds to the active surface of the at least one active polymer layer.

8. The method for manufacturing the biosensor as claimed in claim 7, wherein each of the plurality of capture biomolecules covalently bonds to the plurality of noble metal nanoparticles.

9. The method for manufacturing the biosensor as claimed in claim 6, wherein the plurality of noble metal nanoparticles couples to a covered area of the active surface of the at least one active polymer layer, and wherein the active surface of the at least one active polymer layer further comprises an exposed area to which the plurality of noble metal nanoparticles does not couple.

10. The method for manufacturing the biosensor as claimed in claim 9, further comprising covering the exposed area of the active surface of the at least one active polymer layer by a blocking layer.

11. The method for manufacturing the biosensor as claimed in claim 1, wherein the active surface of the at least one active polymer layer has a functional group selected from a group consisting of an amine group and an ammonium group.

12. The method for manufacturing the biosensor as claimed in claim 11, wherein each of the at least one active polymer layer is formed by a polymer consisting of polyethylenimine, poly(allylamine hydrochloride), poly(β-amino ester), polydiallyldimethylammonium chloride and polyacrylamide.

13. The method for manufacturing the biosensor as claimed in claim 12, wherein the polyethylenimine is a linear polyethylenimine or a branched polyethylenimine.

14. The method for manufacturing the biosensor as claimed in claim 12, wherein the poly(β-amino ester) is a linear poly(β-amino ester) or a branched poly(β-amino ester).
